SEQUENCE LISTING (76 sequences)

SEQ ID NO: 1 - DNA, 933 bp, Malus domestica
atggggagagcaccttgttgtgagaaatctcataccaacaaaggggcatggacgaaggaggaggaccagcgcctcatcgaccacatccgccagcacggcgagggctgctggcgctccctccctaaagccgccgggttgctcaggtgcggcaagagttgcaggttgagatggataaactacctccgccctgacctcaagcgcggcaatttcacacaagaagaagatgaactcatcatcaagcttcatagcttgcttggaaacaaatggtcattgattgcgggccgattgccgggaagaaccgacaacgagatcaaaaactactggaacacacacatcaagcgaaagctcatcagccgcggcctcgaccctcaaacccaccgcccgcttaaccaaaccacaaccgccgctgctgccgccacgcccgcctctcgcttggacctgagaaatcgttcttcgccgtcgtccgcggtatttgatcacaaaaccatcaaaaacaacaaatttgaattgttgaagcaccccaagatggagcatgagtactacaattacaatatagaatcggaggccaattgcagcaccactaccggcagcggcaccacaaccgatgaagaccaaaaacaacagaacaagtacaagtgttctgatctcaacttggatctttcaattgggctagagccgtttcagtccgagccaactcgggcatcgtctgggaactcggccgaatcaagactgcagcggatcatagctccttctaatagtaataataatcatcagttttttgggaaagtgcagtcgactcaggcggtggcggtggctcaggcggtgtgtgtgtgctgccaggttggatttcaaagcagtgaggcatgcagcaattgtcagtgcaccagttacaatggcttctacagatttcacagacctgctttgaatccatag

SEQ ID NO: 2 - protein, 310 aa, Malus domestica
MGRAPCCEKSHTNKGAWTKEEDQRLIDHIRQHGEGCWRSLPKAAGLLRCGKSCRLRWINYLRPDLKRGNFTQEEDELIIKLHSLLGNKWSLIAGRLPGRTDNEIKNYWNTHIKRKLISRGLDPQTHRPLNQTTTAAAAATPASRLDLRNRSSPSSAVFDHKTIKNNKFELLKHPKMEHEYYNYNIESEANCSTTTGSGTTTDEDQKQQNKYKCSDLNLDLSIGLEPFQSEPTRASSGNSAESRLQRIIAPSNSNNNHQFFGKVQSTQAVAVAQAVCVCCQVGFQSSEACSNCQCTSYNGFYRFHRPALNP

SEQ ID NO: 3 - protein, 187 aa, Fragaria x ananassa
MRKPCCEKTETTKGAWSIQEDQKLIDYIQKHGEGCWNSLPKAAGLRRCGKSCRLRWINYLRPDLKRGSFGEDEEDLIIRLHKLLGNRWSLIAGRLPGRTDNEVKNYWNSHLKKKILKTGTTLRPNKPHENNHAPNNKLVKLFNKMDDEVVDEVSSADSAAGCLVPELNLDLTLSIKTSTGMADPQVA

SEQ ID NO: 4 - protein, 257 aa, Arabidopsis thaliana
MGRSPCCEKAHMNKGAWTKEEDQLLVDYIRKHGEGCWRSLPRAAGLQRCGKSCRLRWMNYLRPDLKRGNFTEEEDELIIKLHSLLGNKWSLIAGRLPGRTDNEIKNYWNTHIKRKLLSRGIDPNSHRLINESVVSPSSLQNDVVETIHLDFSGPVKPEPVREEIGMVNNCESSGTTSEKDYGNEEDWVLNLELSVGPSYRYESTRKVSVVDSAESTRRWGSELFGAHESDAVCLCCRIGLFRNESCRNCRVSDVRTH

SEQ ID NO: 5 - protein, 282 aa, Arabidopsis thaliana
MGRSPCCEKAHTNKGAWTKEEDERLVAYIKAHGEGCWRSLPKAAGLLRCGKSCRLRWINYLRPDLKRGNFTEEEDELIIKLHSLLGNKWSLIAGRLPGRTDNEIKNYWNTHIRRKLINRGIDPTSHRPIQESSASQDSKPTQLEPVTSNTINISFTSAPKVETFHESISFPGKSEKISMLTFKEEKDECPVQEKFPDLNLELRISLPDDVDRLQGHGKSTTPRCFKCSLGMINGMECRCGRMRCDVVGGSSKGSDMSNGFDFLGLAKKETTSLLGFRSLEMK

SEQ ID NO: 6 - protein, 269 aa, Arabidopsis thaliana
MGRSPCCEKEHMNKGAWTKEEDERLVSYIKSHGEGCWRSLPRAAGLLRCGKSCRLRWINYLRPDLKRGNFTHDEDELIIKLHSLLGNKWSLIAARLPGRTDNEIKNYWNTHIKRKLLSKGIDPATHRGINEAKISDLKKTKDQIVKDVSFVTKFEETDKSGDQKQNKYIRNGLVCKEERVVVEEKIGPDLNLELRISPPWQNQREISTCTASRFYMENDMECSSETVKCQTENSSSISYSSIDISSSNVGYDFLGLKTRILDFRSLEMK

SEQ ID NO: 7 - protein, 275 aa, Zea mays
MGRSPCCEKAHTNKGAWTKEEDERLVAHIRAHGEGCWRSLPKAAGLLRCGKSCRLRWINYLRPDLKRGNFTEEEDELIVKLHSVLGNKWSLIAGRLPGRTDNEIKNYWNTHIRRKLLSRGIDPVTHRPVTEHHASNITISFETEVAAAARDDKKGAVFRLEDEEEEERNKATMVVGRDRQSQSHSHSHPAGEWGQGKRPLKCPDLNLDLCISPPCQEEEEMEEAAMRVRPAVKREAGLCFGCSLGLPRTADCKCSSSSFLGLRTAMLDFRSLEMK

SEQ ID NO: 8 - protein, 14 aa, Artificial Sequence
  Synthetic peptide
LISRGIDPXXHRXX

SEQ ID NO: 9 - protein, 10 aa, Artificial Sequence
  Synthetic peptide
PDLNLXLXIX

SEQ ID NO: 10 - DNA, 21 bp, Artificial Sequence
  Synthetic oligonucleotide
gggagagcaccttgttgtgag

SEQ ID NO: 11 - DNA, 21 bp, Artificial Sequence
  Synthetic oligonucleotide
gatctcgttgtcggttcttcc

SEQ ID NO: 12 - DNA, 20 bp, Artificial Sequence
  Synthetic oligonucleotide
ggagagcaccttgttgtgag

SEQ ID NO: 13 - DNA, 20 bp, Artificial Sequence
  Synthetic oligonucleotide
actgacaattgctgcatgcc

SEQ ID NO: 14 - DNA, 24 bp, Artificial Sequence
  Synthetic oligonucleotide
ctgatccagaagaagaaacagatg

SEQ ID NO: 15 - DNA, 21 bp, Artificial Sequence
  Synthetic oligonucleotide
tggattcaaagcaggtctgtg

SEQ ID NO: 16 - DNA, 29 bp, Artificial Sequence
  Synthetic oligonucleotide
tgactctagactgatccagaagaagaaac

SEQ ID NO: 17 - DNA, 24 bp, Artificial Sequence
  Synthetic oligonucleotide
atacgagctctggattcaaagcag

SEQ ID NO: 18 - DNA, 22 bp, Artificial Sequence
  Synthetic oligonucleotide
tcacctcttcaaacaacacacc

SEQ ID NO: 19 - DNA, 20 bp, Artificial Sequence
  Synthetic oligonucleotide
tgctctccccatctgtttct

SEQ ID NO: 20 - DNA, 20 bp, Artificial Sequence
  Synthetic oligonucleotide
ggcaagagttgcaggttgag

SEQ ID NO: 21 - DNA, 21 bp, Artificial Sequence
  Synthetic oligonucleotide
gtcgcttgatgtgtgtgttcc

SEQ ID NO: 22 - DNA, 20 bp, Artificial Sequence
  Synthetic oligonucleotide
tcaagcctattgggatttcg

SEQ ID NO: 23 - DNA, 20 bp, Artificial Sequence
  Synthetic oligonucleotide
cagctgacttcctcctcacc

SEQ ID NO: 24 - DNA, 20 bp, Artificial Sequence
  Synthetic oligonucleotide
gatatcgaagccggaaatga

SEQ ID NO: 25 - DNA, 20 bp, Artificial Sequence
  Synthetic oligonucleotide
tgttgactcacgccaacaat

SEQ ID NO: 26 - DNA, 20 bp, Artificial Sequence
  Synthetic oligonucleotide
acaccaaatatggctcctgc

SEQ ID NO: 27 - DNA, 20 bp, Artificial Sequence
  Synthetic oligonucleotide
tttcgttgctgaagtcgttg

SEQ ID NO: 28 - DNA, 20 bp, Artificial Sequence
  Synthetic oligonucleotide
aatgggagtggagtctgtgg

SEQ ID NO: 29 - DNA, 20 bp, Artificial Sequence
  Synthetic oligonucleotide
agttggagctggcctcagta

SEQ ID NO: 30 - DNA, 20 bp, Artificial Sequence
  Synthetic oligonucleotide
aaggccgttacatttgttcg

SEQ ID NO: 31 - DNA, 20 bp, Artificial Sequence
  Synthetic oligonucleotide
gcccttgaactttgtgggta

SEQ ID NO: 32 - DNA, 20 bp, Artificial Sequence
  Synthetic oligonucleotide
agctccactcggaacttcaa

SEQ ID NO: 33 - DNA, 20 bp, Artificial Sequence
  Synthetic oligonucleotide
aacccgccctaaatatgtcc

SEQ ID NO: 34 - DNA, 20 bp, Artificial Sequence
  Synthetic oligonucleotide
caatttggcctcaaacacct

SEQ ID NO: 35 - DNA, 20 bp, Artificial Sequence
  Synthetic oligonucleotide
tcaacaccaagtgcaagctc

SEQ ID NO: 36 - DNA, 20 bp, Artificial Sequence
  Synthetic oligonucleotide
gttgcaacccctgtcaactt

SEQ ID NO: 37 - DNA, 20 bp, Artificial Sequence
  Synthetic oligonucleotide
cacgaccaaacctgttcctt

SEQ ID NO: 38 - DNA, 20 bp, Artificial Sequence
  Synthetic oligonucleotide
acaacacccacccttctgag

SEQ ID NO: 39 - DNA, 20 bp, Artificial Sequence
  Synthetic oligonucleotide
tgcagcaagggctagtaggt

SEQ ID NO: 40 - DNA, 23 bp, Artificial Sequence
  Synthetic oligonucleotide
ctacaaagtcatcgtccagacat

SEQ ID NO: 41 - DNA, 21 bp, Artificial Sequence
  Synthetic oligonucleotide
tgggatgacatggagaagatt

SEQ ID NO: 42 - DNA, 20 bp, Artificial Sequence
  Synthetic oligonucleotide
aatgatcggaatggaagctg

SEQ ID NO: 43 - DNA, 20 bp, Artificial Sequence
  Synthetic oligonucleotide
tggtaccaccactgaggaca

SEQ ID NO: 44 - DNA, 20 bp, Artificial Sequence
  Synthetic oligonucleotide
gaaatcctccgatccagtga

SEQ ID NO: 45 - DNA, 20 bp, Artificial Sequence
  Synthetic oligonucleotide
caacgttgacaacatcaggc

SEQ ID NO: 46 - DNA, 20 bp, Artificial Sequence
  Synthetic oligonucleotide
agaaaagccttgtggaagca

SEQ ID NO: 47 - DNA, 20 bp, Artificial Sequence
  Synthetic oligonucleotide
acttggtccaaaattgcagg

SEQ ID NO: 48 - DNA, 20 bp, Artificial Sequence
  Synthetic oligonucleotide
acagggtgaagtggtccaag

SEQ ID NO: 49 - DNA, 20 bp, Artificial Sequence
  Synthetic oligonucleotide
ccttggttaaggcctccttc

SEQ ID NO: 50 - DNA, 20 bp, Artificial Sequence
  Synthetic oligonucleotide
tccaagaatactggcccaag

SEQ ID NO: 51 - DNA, 20 bp, Artificial Sequence
  Synthetic oligonucleotide
ctcacaactctcggatgcaa

SEQ ID NO: 52 - DNA, 20 bp, Artificial Sequence
  Synthetic oligonucleotide
gaacttgaagggaaaagggg

SEQ ID NO: 53 - DNA, 20 bp, Artificial Sequence
  Synthetic oligonucleotide
tccctgtaggagggaggatt

SEQ ID NO: 54 - DNA, 20 bp, Artificial Sequence
  Synthetic oligonucleotide
tcccatcatgcgatcatcta

SEQ ID NO: 55 - DNA, 20 bp, Artificial Sequence
  Synthetic oligonucleotide
atggcttctttgtcacgtcc

SEQ ID NO: 56 - DNA, 20 bp, Artificial Sequence
  Synthetic oligonucleotide
tcaaggtcctttacgccatc

SEQ ID NO: 57 - DNA, 20 bp, Artificial Sequence
  Synthetic oligonucleotide
acgaacctgcttctctttgg

SEQ ID NO: 58 - DNA, 20 bp, Artificial Sequence
  Synthetic oligonucleotide
tggcgttgaagctcatactg

SEQ ID NO: 59 - DNA, 20 bp, Artificial Sequence
  Synthetic oligonucleotide
tttcaagggtgtccccaata

SEQ ID NO: 60 - DNA, 20 bp, Artificial Sequence
  Synthetic oligonucleotide
gagtgcattggatgcctttt

SEQ ID NO: 61 - DNA, 20 bp, Artificial Sequence
  Synthetic oligonucleotide
ccagctccattaggtccttg

SEQ ID NO: 62 - DNA, 20 bp, Artificial Sequence
  Synthetic oligonucleotide
catttgactttcccaaacgc

SEQ ID NO: 63 - DNA, 20 bp, Artificial Sequence
  Synthetic oligonucleotide
attgggcttttgagttgtgc

SEQ ID NO: 64 - DNA, 20 bp, Artificial Sequence
  Synthetic oligonucleotide
tgttcccacttgggatgata

SEQ ID NO: 65 - DNA, 23 bp, Artificial Sequence
  Synthetic oligonucleotide
tgcacctatactctgttagtggc

SEQ ID NO: 66 - DNA, 20 bp, Artificial Sequence
  Synthetic oligonucleotide
ccaggagtgcaagtgactga

SEQ ID NO: 67 - DNA, 20 bp, Artificial Sequence
  Synthetic oligonucleotide
accaccaagcgttaaccaag

SEQ ID NO: 68 - DNA, 20 bp, Artificial Sequence
  Synthetic oligonucleotide
cctcagaacatcaccccagt

SEQ ID NO: 69 - DNA, 20 bp, Artificial Sequence
  Synthetic oligonucleotide
accgtgtaacgccttgtttc

SEQ ID NO: 70 - DNA, 20 bp, Artificial Sequence
  Synthetic oligonucleotide
ttttcgtggatgctgacaag

SEQ ID NO: 71 - DNA, 20 bp, Artificial Sequence
  Synthetic oligonucleotide
gggtaattccatcaccaacg

SEQ ID NO: 72 - DNA, 20 bp, Artificial Sequence
  Synthetic oligonucleotide
cgaagacattggctgaggat

SEQ ID NO: 73 - DNA, 20 bp, Artificial Sequence
  Synthetic oligonucleotide
ttgggtatgtttcagcacca

SEQ ID NO: 74 - DNA, 56 bp, Artificial sequence
  Synthetic oligonucleotide
tctctctctctctctctctctctctctctctctatatatatatatatatatatata

SEQ ID NO: 75 - DNA, 10 bp, Artificial sequence
  Synthetic oligonucleotide
gtgtgtgtgt

SEQ ID NO: 76 - protein, 20 aa, Artificial sequence
  Synthetic peptide
XLXXXXXXLXXXXXXLXXXR

(19) United States
(12) Patent Application Publication
(10) Pub. No.: US 2014/0250550 A1
(43) Pub. Date: Sep. 4, 2014

(54) COMPOSITIONS AND METHODS FOR MODULATING ANTHOCYANIN ACCUMULATION AND PISTIL DEVELOPMENT

(71) Applicant: The Board of Trustees of the University of Illinois, Urbana, IL (US)

(72) Inventors: Schuyler S. Korban, Champaign, IL (US); Yeupeng Han, Wuchan (CN); Sornkanok Vimolmangkang, Bangkok (TH)

(73) Assignee: The Board of Trustees of the University of Illinois, Urbana, IL (US)

(21) Appl. No.: 14/193,455

(22) Filed: Feb. 28, 2014

Related U.S. Application Data
(60) Provisional application No. 61/771,811, filed on Mar. 2, 2013.

(51) Int. Cl. C07K 14/415; C12N 15/82

(52) U.S. Cl. CPC C07K 14/415; C12N 15/8261; C12N 15/8243
USPC 800/290; 536/23.6; 530/370; 435/320.1; 800/298; 800/278

(57) ABSTRACT

MdMYB3 nucleic acids and polypeptides are provided for use in modulating the accumulation of anthocyanins and flavonols as well as the length of styles and peduncles of transgenic flowers.

This application claims benefit of priority from U.S. Provisional Patent Application Serial. No. 61/771,881, filed Mar. 3, 2013, the content of which is incorporated herein by reference in its entirety.

This invention was made with government support under contract number AG 2009-51181-06023 awarded by the United States Department of Agriculture-National Institute of Food and Agriculture-Specialty Crop Research Initiative. The government has certain rights in the invention.

### INTRODUCTION

### Background

Skin color is an important determinant of apple fruit quality. Generally, consumers prefer red-skinned apples as they are perceived to be associated with better taste and flavor (King & Cliff (2002) J. Amer. Pomol. Soc. 56:223-229). Coloration of apple fruit is attributed to accumulation of anthocyanins, a class of plant flavonoid metabolites. Flavonoids are ubiquitous in plants, and play important roles throughout plant growth, including UV protection, disease resistance, herbivore defense, and providing flowers and seeds with pigmentation to attract pollinators and seed dispersers (Kevan, et al. (1996) Trends Plant Sci. 1:280-284; Schaefer, et al. (2004) Trends Ecol. Evol. 19:577-584). More importantly, there is increasing evidence that flavonoids benefit human health such as lowering the incidence of cardiovascular disease, obesity, diabetes, pulmonary disease, and cancer (Nijveldt, et al. (2001) Amer. J. Clin. Nutr. 74:418-425; Boyer & Liu (2004) Nutr. J. 3:1-45; Ruxton, et al. (2006) Int. J. Food Sci. Nutr. 57:249-272; Gerhauser (2008) Planta Med. 74:1608-1624; Prasad, et al. (2010) Planta Med. 76:1044-1063).

The biosynthetic pathway of anthocyanins has been analyzed and anthocyanin pathway genes have been isolated and characterized in a variety of model plants such as petunia, snapdragon, and Arabidopsis (Winkel-Shirley (2001) Plant Physiol. 126:485-493; Tanaka, et al. (2008) Plant J. 54:733-749). Anthocyanin biosynthesis is genetically determined by structural and regulatory genes. The structural genes are regulated at the transcriptional level by regulatory genes, and thus plant pigmentation patterns are mainly controlled by the expression profiles of regulatory genes (Holton & Cornish (1995) Plant Cell 7:1071-1083; Grotewold (2006) Annu. Rev. Plant Biol. 57:761-780).

Three transcription factors (TFs), the basic helix-loop-helix (bHLH), R2R3MYB, and WD40 proteins, predominantly regulate genes in the anthocyanin biosynthesis pathway across various plant species (Stracke, et al. (2001) Curr. Opin. Plant Biol. 4:447-456; Allan, et al. (2008) Trends Plant Sci. 13:99-102). MYB TFs have been reported to play diverse functions in controlling pathways such as secondary metabolism, development, signal transduction, and disease resistance in plants (Allan, et al. (2008) Trends Plant Sci. 13:99-102). They are classified by the numbers of highly conserved imperfect repeats in the DNA-binding domain, and are composed of either single or multiple repeats. Among these MYB TFs, the class of two-repeats (R2R3) is deemed the largest, with 339 TFs reported in Arabidopsis (Feller, et al. (2011) Plant J. 66:94-116), and it is associated with the anthocyanin biosynthesis pathway.

Regulation of R2R3MYB TFs can occur at different steps of the anthocyanin biosynthesis pathway. For example, R2R3MYB TFs in perilla (Perilla frutescens) control the transcription of all structural genes involved in anthocyanin biosynthesis (Saito & Yamazaki (2002) New Phytol. 155:9-23). MYBA in grape (Vitis vinifera) specifically regulates genes down-stream of anthocyanin production, but not those of earlier steps (Kobayashi, et al. (2002) Planta 215:924-933). As with other transcription factors, regulation of R2R3MYB TFs could serve to either activate or repress expression of these genes. For example, MYB TFs such as Arabidopsis PAP1, AtPAP2, AtMYB113, and AtMYB114 (Borevitz, et al. (2000) Plant Cell 12:2383-2393; Gonzalez, et al. (2008) Plant J. 53:814-827), grape VvMYB1a (Kobayashi, et al. (2004) Science 304:982), and Gerbera hybrid GhMYB10 (Elomaa, et al. (2003) Plant Physiol. 133:1831-1842) positively regulate anthocyanin biosynthesis. Suppression of the flavonoid accumulation has been observed in transgenic plants overexpressing strawberry FaMYB1 (Aharoni, et al. (2001) Plant J. 28:319-332), Antirrhinum AmMYB308 (Tamagnone, et al. (1998) Plant Cell 10:135-154), Arabidopsis AtMYB4 (Jin, et al. (2000) EMBO J. 19:6150-6161), and Arabidopsis AtMYBL2 which encode a single-repeat R3-MYB protein (Dubos, et al. (2010) Trends Plant Sci. 15:573-581; Matsui, et al. (2008) Plant J. 55:954-967). Furthermore, overexpression of Antirrhinum AmMYB308 in transgenic tobacco results in smaller flowers, elongated styles, protruded stigmas, low levels of anthocyanin and infrequent self-pollination (Tamagnone, et al. (1998) Plant Cell 10:135-154).

Several studies have reported on the characterization of structural and regulatory genes involved in fruit coloration in apple (Malus×domestica Borkh.). For example, induction of most structural genes in the anthocyanin biosynthesis pathway can significantly increase accumulation of anthocyanin in apple skin (Honda, et al. (2002) Plant Physiol. Biochem. 40:955-962). Three transcription factors, MdMYB10, MdMYB1, and MdMYBA have been isolated and characterized in apple (Takos, et al. (2006) Plant Physiol. 142:1216-1232; Ban, et al. (2007) Plant Cell Physiol. 48:958-970; Espley, et al. (2007) Plant J. 49:414-427). Of the three TFs, MdMYB10 is responsible for red flesh coloration, while MdMYB1 and MdMYBA control red skin coloration of apple fruit. The three MdMYB genes are almost identical in nucleotide sequences, and have been subsequently reported to be of different alleles on linkage group 9 (Chagne, et al. (2007) BMC Genomics 8:212; Lin-Wang, et al. (2010) BMC Plant Biol. 10:50). In addition, it has been reported that the red-flesh cortex phenotype of apple fruit is associated with enhanced expression of MYB110a, a paralog of MYB10, and functional analysis of MYB110a in tobacco has revealed that it is involved in up-regulation of anthocyanin biosynthesis (Chagne, et al. (2013) Plant Physiol. 161:225-239). Apple fruits vary considerably in color, ranging from yellow, green, or red, along with varied differences in red color pigmentation patterns. Thus, it is unlikely that apple fruit skin red coloration is simply controlled by a single locus.

### SUMMARY OF THE INVENTION

This invention is a purified nucleic acid molecule composed of (a) a nucleotide sequence encoding a transcription factor having the amino acid sequence of SEQ ID NO:2; (b) the nucleotide sequence of SEQ ID NO:1 or a sequence at least 75% identical thereto; or (c) a nucleotide sequence complementary to a nucleotide sequence of (a) or (b). A recombinant vector, an expression vector, or a transgenic plant or plant part is also provided as is a progeny or seed of said transgenic plant. This invention also provides a purified transcription factor polypeptide having the amino acid sequence of SEQ ID NO:2, or a polypeptide at least 75% identical thereto, and a method for modifying the color, length of peduncles, or length of styles of a plant.

### BRIEF DESCRIPTION OF THE DRAWINGS

FIG. 1 depicts an illustrative embodiment of the comparisons of deduced amino acid sequences of MdMYB3 (SEQ ID NO:2) in apple and R2R3MYB proteins from strawberry (FaMYB1, SEQ ID NO:3), Arabidopsis (AtMYB3, SEQ ID NO:4; AtMYB4, SEQ ID NO:5; AtMYB7, SEQ ID NO:6) and maize (ZmMYB31, SEQ ID NO:7) using ClustalW2 program. R2 and R3 repeats are indicated with brackets. The bHLH motif is indicated in bold, while C1 (SEQ ID NO:8) and C2 (SEQ ID NO:9) motifs are underlined. Conserved sequences with 100%, 80%, and 60% identity are marked with asterisks, two dots, and one dot, respectively.

FIG. 2 depicts the deduced functionality of MdMYB3 in apple based on analysis of its ectopic expression in tobacco flowers. The possible pathway that MdMYB3 may active the transcription of NtAn2 (anthocyandin) and thus induce flavonoid pathway genes, Chalcone Flavanone Isomerase (CHI), Chalcone Synthase (CHS), UDP Glucose-Flavonoid 3-O-Glucosyl Transferase (UFGT), and Flavonol Synthase (FLS), is indicated by dotted lines.

FIG. 3 depicts an illustrative embodiment of the expression profiles of MdMYB3 in various tissues of apple cultivars Red Delicious (RD) and Golden Delicious (GD) using qRT-PCR. Abbreviations are listed as followings: Fw1: Flower buds at the pink stage; Fw2: flower buds at the balloon stage; Fw3: flowers at full bloom; Ft1: 9 days after pollination (DAP); Ft2: 16 DAP; Ft3: 44 DAP; Ft4: 104 DAP; Ft5: 145 DAP; Ft6: 166 DAP; RD: ‘Red delicious’; GD: ‘Golden Delicious’. Normalization was made to the expression of actin gene and values are the average of three technical replicates.

FIG. 4 depicts expression analysis of phenylpropanoid and flavonoid pathway genes in flowers of T2 transgenic tobacco lines carrying MdMYB3 using qRT-PCR. Normalization was made to the expression of actin gene and values are the average of three technical replicates, and the transcript levels in transgenic flowers were quantified relative to those present in wild-type flowers. The abbreviations were listed as follows: CHI: chalcone isomerase; CHS: chalcone synthase; F3H: flavonoid 3-hydroxylase; F3′H: flavonoid 3′-hydroxylase; FLS: flavonol synthase; DFR: dihydroflavonol reductase; LAR: leucoanthocyanidin reductase; ANS: anthocyanidin synthase; UFGT: glucose transferase; ANR: anthocyanidin reductase; C4H: cinnamate-4-hydroxylase; 4CL: 4-coumaroyl-CoA ligase; PAL: phenylalanine ammonia lyase; COMT: caffeoyl-CoA o-methyltransferase; CAD: cinnamyl alcohol dehydrogenase.

### DETAILED DESCRIPTION OF THE INVENTION

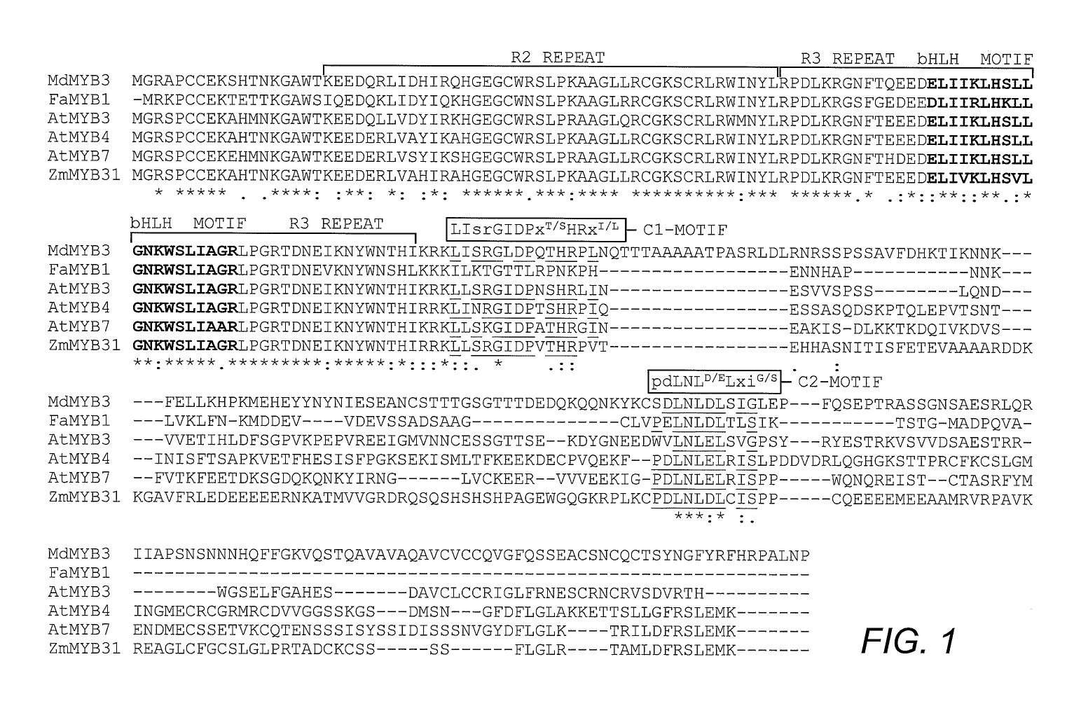

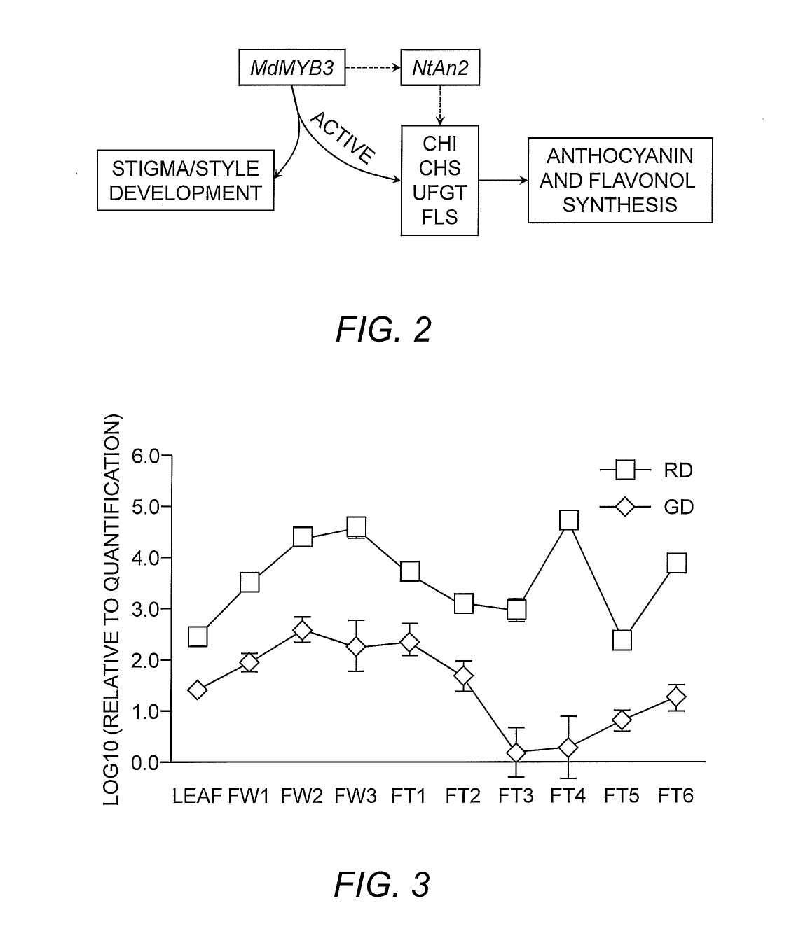

Isolation and characterization of MYB TFs associated with anthocyanin biosynthesis is an important key step towards understanding and manipulating fruit coloration. As described herein, a MYB TF, designated MdMYB3 (FIG. 1), has now been identified using an apple expressed sequence tag (EST) database (Gasic, et al. (2009) Plant Genome 2:23-38) and a BAC-based physical map of the apple genome (Han, et al. (2011) J. Exp. Bot. 62:5117-5130). The MdMYB3 gene shows higher levels of expression in exocarp of red-skinned apple cultivars than that of yellowish-green skinned apple cultivars. Transgenic flowers overexpressing MdMYB3 accumulate higher levels of anthocyanin, resulting in increased color pigmentation, and have longer peduncles and styles when compared with those of wild-type flowers. Overexpression of MdMYB3 in tobacco flowers significantly repressed transcription of genes involved in the lignin biosynthesis pathway such as C4H and 4CL2; moreover, it also severely inhibited expression of the CAD gene involved in monolignol biosynthesis (FIG. 2). However, expression of NtCOMT in flowers of transgenic lines overexpressing MdMYB3 is significantly higher than that in wild-type plants (FIG. 2). These results indicate that MdMYB3 not only regulates anthocyanin biosynthesis, but is also involved in flower and pistil development. As such, this invention provides nucleic acids encoding MdMYB3, MdMYB3 polypeptides, and methods of using the same to enhance accumulation of anthocyanins/flavonols and increase the length of peduncles and styles.

As discussed herein, a MYB TF associated with anthocyanin biosynthesis and flower and pistil development in apple has now been identified. This MYB TF is described herein as MdMYB3. Nucleic acids encoding MdMYB3 are set forth herein in SEQ ID NO:1. For the purposes of the present invention, the terms “polynucleotide(s),” “nucleic acid sequence(s),” “nucleotide sequence(s),” “nucleic acid(s),” and “nucleic acid molecule” are used interchangeably and refer to nucleotides, either ribonucleotides or deoxyribonucleotides or a combination of both, in a polymeric unbranched form of any length. An MdMYB3 nucleic acid of this invention includes that set forth in SEQ ID NO:1, as well as splice variants, allelic variants, variants generated by directed evolution, and orthologs or paralogs thereof.

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be man-made. Methods for predicting and isolating such splice variants are well known in the art (see, for example, Foissac & Schiex (2005) BMC Bioinformatics 6:25).

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

Gene shuffling or directed evolution is the process of iterations of DNA shuffling followed by appropriate screening and/or selection to generate variants of nucleic acids or portions thereof encoding proteins having a modified biological activity (Castle, et al. (2004) Science 304(5674):1151-4; U.S. Pat. No. 5,811,238; U.S. Pat. No. 6,395,547).

In certain embodiments, splice variants, allelic variants, variants generated by directed evolution, and orthologs or paralogs of a nucleic acid encoding MdMYB3 share between 75.0 and 99.9% sequence identity with SEQ ID NO:1. In other embodiments, variants, orthologs or paralogs of a nucleic acid encoding MdMYB3 are at least 75%, 85%, 90%, 95%, 97%, or 99% identical to SEQ ID NO:1.

Methods of alignment of sequences for comparison are well-known in the art. Optimal alignment of sequences for comparison may be conducted by the local homology algorithm of Smith & Waterman (1981) Adv. Appl. Math. 2:482; by the homology alignment algorithm of Needleman & Wunsch (1970) J. Mol. Biol. 48:443; by the search for similarity method of Pearson & Lipman (1988) Proc. Natl. Acad. Sci. 85:2444; by computerized implementations of these algorithms, including, but not limited to: CLUSTAL in the PC/Gene program by Intelligenetics (Mountain View, Calif.); GAP, BESTFIT, BLAST, FASTA, and TFASTA in the Wisconsin Genetics Software Package, Genetics Computer Group (GCG; Madison, Wis.); the CLUSTAL program is well described by Higgins & Sharp (1988) Gene 73:237-244; Higgins & Sharp (1989) CABIOS 5:151-153; and Corpet, et al. (1988) Nucl. Acids Res. 16:10881-90. The BLAST family of programs that can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST suite of programs using default parameters. Altschul, et al. (1997) Nucl. Acids Res. 25:3389-3402. Software for performing BLAST analyses is publicly available through the National Center for Biotechnology-Information.

As used herein, percent sequence identity means the value determined by comparing two optimally aligned sequences over a comparison window, wherein the portion of the polynucleotide or polypeptide sequence in the comparison window may include additions or deletions (i.e., gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As will be apparent to those skilled in the art, SEQ ID NO:1 represents the coding sequence of MdMYB3. A “coding sequence” is a nucleic acid sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. Therefore, in addition to SEQ ID NO:1, the present invention also includes the gene encoding MdMYB3, i.e., the coding sequence as well as any intron, 3′ untranslated region, 5′ untranslated region, promoter or terminator sequences of the MdMYB3 gene.

Nucleic acids that hybridize to SEQ ID NO:1 are also included in this invention. The term “hybridize” or “hybridization” refers to a process wherein substantially homologous complementary nucleotide sequences anneal to each other. The hybridization process can occur entirely in solution, i.e., both complementary nucleic acids are in solution. The hybridization process can also occur with one of the complementary nucleic acids immobilized to a matrix such as magnetic beads, SEPHAROSE beads, or any other resin. The hybridization process can furthermore occur with one of the complementary nucleic acids immobilized to a solid support such as a nitro-cellulose or nylon membrane or immobilized by, e.g., photolithography to, for example, a siliceous glass support (the latter known as nucleic acid arrays or microarrays or as nucleic acid chips). In order to allow hybridization to occur, the nucleic acid molecules are generally thermally or chemically denatured to melt a double strand into two single strands and/or to remove hairpins or other secondary structures from single stranded nucleic acids.

In certain embodiments, a nucleic acid that hybridizes to SEQ ID NO:1, does so under stringent conditions. “Stringency” refers to the conditions under which a hybridization takes place. The stringency of hybridization is influenced by conditions such as temperature, salt concentration, ionic strength and hybridization buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridization conditions are typically used for isolating hybridizing sequences that have high sequence similarity to the target nucleic acid sequence. However, nucleic acids may deviate in sequence and still encode a substantially identical polypeptide, due to the degeneracy of the genetic code. Therefore, medium stringency hybridization conditions may sometimes be needed to identify such nucleic acid molecules.

The Tₘ is the temperature under defined ionic strength and pH, at which 50% of the target sequence hybridizes to a perfectly matched probe. The Tₘ is dependent upon the solution conditions and the base composition and length of the probe. For example, longer sequences hybridize specifically at higher temperatures. The maximum rate of hybridization is obtained from about 16° C. up to 32° C. below Tₘ. The presence of monovalent cations in the hybridization solution reduce the electrostatic repulsion between the two nucleic acid strands thereby promoting hybrid formation; this effect is visible for sodium concentrations of up to 0.4 M (for higher concentrations, this effect may be ignored). Formamide reduces the melting temperature of DNA-DNA and DNA-RNA duplexes with 0.6 to 0.7° C. for each percent formamide, and addition of 50% formamide allows hybridization to be performed at 30 to 45° C., though the rate of hybridization will be lowered. Base pair mismatches reduce the hybridization rate and the thermal stability of the duplexes. On average and for large probes, the Tₘ decreases about 1° C. per % base mismatch.

Non-specific binding may be controlled using any one of a number of known techniques such as, for example, blocking the membrane with protein containing solutions, additions of heterologous RNA, DNA, and SDS to the hybridization buffer, and treatment with RNase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridization and which will either maintain or change the stringency conditions.

Besides the hybridization conditions, specificity of hybridization typically also depends on the function of post-hybridization washes. To remove background resulting from non-specific hybridization, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridization stringency. A positive hybridization gives a signal that is at least twice of that of the background. Generally, suitable stringent conditions for nucleic acid hybridization assays or gene amplification detection procedures are as set forth above. More or less stringent conditions may also be selected. The skilled artisan is aware of various parameters which may be altered during washing and which will either maintain or change the stringency conditions.

For example, typical high stringency hybridization conditions for DNA hybrids longer than 50 nucleotides encompass hybridization at 65° C. in 1×SSC (0.15 M NaCl and mM sodium citrate) or at 42° C. in 1×SSC and 50% formamide, followed by washing at 65° C. in 0.3×SSC. Examples of medium stringency hybridization conditions for DNA hybrids longer than 50 nucleotides encompass hybridization at 50° C. in 4×SSC or at 40° C. in 6×SSC and 50% formamide, followed by washing at 50° C. in 2×SSC. In addition to SSC and optional formamide, the hybridization solution and wash solutions may include 5×Denhardt's reagent, 0.5-1.0% SDS, 100 μg/ml denatured, fragmented salmon sperm DNA, 0.5% sodium pyrophosphate. The length of the hybrid is the anticipated length for the hybridizing nucleic acid. When nucleic acids of known sequence are hybridized, the hybrid length may be determined by aligning the sequences and identifying the conserved regions described herein. In certain embodiments of this invention, a nucleic acid that hybridizes to a nucleic acid of SEQ ID NO:1, does so under high stringency hybridization conditions.

For the purposes of defining the level of stringency, reference can be made to Sambrook, et al. (2001) Molecular Cloning: a Laboratory Manual, 3^{rd} Edition, Cold Spring Harbor Laboratory Press, CSH, New York or to Current Protocols in Molecular Biology, John Wiley & Sons, NY (1989 and yearly updates).

MdMYB3 polypeptides are also included in this invention. The terms “polypeptide” and “protein” are used interchangeably herein and refer to amino acids in a polymeric form of any length, linked together by peptide bonds. In some embodiments, the MdMYB3 polypeptide is set forth herein in SEQ ID NO:2. In other embodiments, the MdMYB3 polypeptide is a splice variant, allelic variant, variant generated by directed evolution, ortholog or paralog of SEQ ID NO:2. In particular embodiments, a variant, ortholog or paralog of SEQ ID NO:2 shares between 75.0 and 99.9% sequence identity with SEQ ID NO:2. In other embodiments, variants, orthologs or paralogs of a MdMYB3 polypeptide are at least 75%, 85%, 90%, 95%, 97%, or 99% identical to SEQ ID NO:2.

Homologs and variants of the MdMYB3 polypeptide of the invention are also included. “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. While a deletion refers to removal of one or more amino acids from a protein, an insertion refers to one or more amino acid residues being introduced into a predetermined site in a protein. Insertions may include N-terminal and/or C-terminal fusions as well as intra-sequence insertions of single or multiple amino acids. Generally, insertions within the amino acid sequence will be smaller than N- or C-terminal fusions, and are in the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, c-myc epitope, FLAG-epitope, lacZ, CMP (calmodulin-binding peptide), HA epitope, protein C epitope, VSV epitope and fluorescent proteins such as green fluorescent protein.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see, for example, Creighton (1984) Proteins. W.H. Freeman and Company (Eds)).

Amino acid substitutions, deletions and/or insertions may readily be made using peptide synthetic techniques well-known in the art, such as solid phase peptide synthesis and the like, or by recombinant DNA manipulation. Methods for the manipulation of DNA sequences to produce substitution, insertion or deletion variants of a protein are well-known in the art. For example, techniques for making substitution mutations at predetermined sites in DNA are well-known to those skilled in the art and include M13 mutagenesis, T7-Gen in vitro mutagenesis (USB, Cleveland, Ohio), QUICKCHANGE Site Directed mutagenesis (Stratagene, San Diego, Calif.), PCR-mediated site-directed mutagenesis or other site-directed mutagenesis protocols.

“Derivatives” of the MdMYB3 polypeptide include peptides, oligopeptides, polypeptides which may, compared to the amino acid sequence of the naturally-occurring form of the protein include substitutions of amino acids with non-naturally occurring amino acid residues, or additions of non-naturally occurring amino acid residues. “Derivatives” of the MdMYB3 polypeptide also encompass peptides, oligopeptides, polypeptides which include naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also include one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS₆ or thioredoxin (for a review of tagging peptides, see Terpe (2003) Appl. Microbiol. Biotechnol. 60:523-533).

The present invention also provides antibodies specific for MdMYB3. Such antibodies can be monoclonal or polyclonal prepared by conventional methods. Antibodies that specifically bind MdMYB3 desirably do not bind to other, similar transcription factors and can be used in the detection of MdMYB3 as well as in inhibiting the activity of MdMYB3.

MdMYB3 nucleic acids and polypeptides of the present invention can be isolated and/or purified, meaning that the nucleic acid or polypeptide is substantially or essentially free from components that normally accompany or interact with it as found in its naturally occurring environment. The purified material optionally includes material not found with the material in its natural environment; or if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes a purified nucleic acid if it is altered, or if it is transcribed from DNA which has been altered, by means of human intervention performed within the cell from which it originates.

In particular embodiments, the MdMYB3 nucleic acids and polypeptides of the invention are recombinant. As used herein, “recombinant” refers to an organism, cell, nucleic acid or protein formed by recombination. Thus, for example, recombinant nucleic acids are molecules formed by laboratory methods of molecular cloning, which create molecules that are not found in biological organisms. In this respect, a cDNA or complementary DNA is a recombinant nucleic acid as it is synthesized in the laboratory from an mRNA template. Similarly, a recombinant polypeptide is a protein prepared by recombinant DNA technology and typically refers to fusion proteins or tagged proteins.

MdMYB3 nucleic acids and polypeptides find use in altering the levels of anthocyanin as well as the length of peduncles and styles in plants. Alterations in the levels of anthocyanin and length of peduncles and styles in plants is achieved by modulating the expression, levels or activity of MdMYB3 nucleic acids and/or MdMYB3 polypeptides. The term “modulation” or “modulating” means in relation to expression or gene expression, a process in which the expression level is changed by said gene expression in comparison to a control plant, the expression level may be increased or decreased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to altered levels of anthocyanin and/or length of peduncles and styles in plants.

In some embodiments, MdMYB3 is overexpressed to increase the levels of anthocyanin and increase the length of peduncles and styles in plants. “Overexpression,” “overexpressed” or “increased expression” means any form of expression that elevated over the expression level in a control plant or wild-type plant, e.g., a plant of the same species or even of the same variety as the plant being assessed. Methods for increasing expression of genes or gene products are well-documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see U.S. Pat. No. 5,565,350 or WO 93/22443), or isolated promoters may be introduced into a plant cell in the proper orientation and distance from a nucleic acid of the present invention so as to control the expression of the nucleic acid.

Depending on the cell, organ or timing of expression required, overexpression of MdMYB3 can be controlled by any of a number of promoters. The term “promoter” typically refers to a nucleic acid control sequence located upstream from the transcriptional start of a gene and which is involved in recognizing and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e., upstream activating sequences, enhancers and silencers), which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

In certain embodiments, the promoter is a plant promoter. A “plant promoter” includes regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g., from the plant which is transformed with the nucleic acid sequence to be expressed. Independent of the source of the promoter, the MdMYB3 nucleic acid must be linked operably to or include a suitable promoter which expresses the gene at the right point in time and with the required spatial expression pattern. The term “operably linked” refers to a functional linkage between the promoter sequence and the gene of interest, such that the promoter sequence is able to initiate transcription of the gene of interest. Suitable promoters of use in this invention can be constitutive, cell-specific, inducible, tissue-specific or organ-specific promoter, or developmentally-regulated promoter.

The term “constitutive” will be known by those skilled in the art to indicate that expression is observed predominantly throughout the plant, albeit not necessarily in every cell, tissue or organ under all conditions. In the present context, a preferred constitutive promoter is one which confers a high level of ectopic expression of MdMYB3 predominantly throughout the plant, albeit not necessarily in every cell, tissue or organ under all conditions. Examples of constitutive promoters include, but are not limited to, actin, CaMV 35S (Odell, et al. (1985) Nature 313:810-12), CaMV 19S, octopine synthase (Koncz, et al. (1984) EMBO J. 3:1029-37), nopaline synthase (Depicker, et al. (1982) J. Mol. Appl. Genet. 1:561-73), gos2 (de Pater, et al. (1992) Plant J. 2:837-44) and UBQ1 (Callis, et al. (1990) J. Biol. Chem. 265:12486-93) promoters.

The term “cell-specific” refers to expression that is predominantly in a particular plant cell or plant cell-type, albeit not necessarily exclusively in that plant cell or plant cell-type. Similarly, the term “tissue-specific” indicates that expression is predominantly in a particular plant tissue or plant tissue-type, albeit not necessarily exclusively in that plant tissue or plant tissue-type. Likewise, the term “organ-specific” indicates that expression is predominantly in a particular plant organ albeit not necessarily exclusively in that plant organ. Tissue-specific or organ-specific promoters include, e.g., flower-specific, seed-specific, root specific and green tissue-specific promoters.

Examples of flower-specific promoters include, but are not limited to, the CHS promoter (Liu, et al. (2011) Plant Cell Rep. 30:2187-94; Kobayashi, et al. (1998) Plant Sci. 131:173-80; Bing-you, et al. (2010) For. Stud. China 12:201-205); petal-specific promoters (U.S. Pat. No. 7,217,859); the EPSPS promoter (Benfey & Chua (1989) Science 244:174-81); APETALA3 promoter (Hill, et al. (1998) Development 125:1711-21); as well as the flower-specific promoters from Brassica (Geng, et al. (2009) African J. Biotech. 8:5193-5200).

Examples of root-specific promoters include RCc3 (Xu, et al. (1995) Plant Mol. Biol. 27(2):237-48); Arabidopsis PHT1 (Kovama, et al. (2005) J. Biosci. Bioeng. 99:38-42; Mudge, et al. (2002) Plant J. 31:341); Medicago phosphate transporter (Xiao, et al. (2006) Plant Biol. (Stuttg.) 8:439-49); Arabidopsis Pyk10 (Nitz, et al. (2001) Plant Sci. 161(2):337-346); root-expressible genes (Tingey, et al. (1987) EMBO J. 6:1); tobacco root-specific genes (Conkling, et al. (1990) Plant Physiol. 93:1203); B. napes G1-3b gene (U.S. Pat. No. 5,401,836) and the like.

Seed-specific promoters may be active during seed development and/or during germination. A seed-specific promoter may be endosperm and/or aleurone and/or embryo specific. Examples of seed-specific promoters, but are not limited to, Brazil Nut albumin (Pearson, et al. (1992) Plant Mol. Biol. 18:235-245); legumin (Ellis, et al. (1988) Plant Mol. Biol. 10:203-214); glutelin (rice) (Takaiwa, et al. (1986) Mol. Gen. Genet. 208:15-22); zein (Matzke, et al. (1990) Plant Mol. Biol. 14(3):323-32); napA (Stalberg, et al. (1996) Planta 199:515-519); wheat SPA (Albani, et al. (1997) Plant Cell 9:171-184); barley Itrl promoter (Diaz, et al. (1995) Mol. Gen. Genet. 248(5):592-8); barley DOF (Mena, et al. (1998) Plant J. 116(1):53-62); sunflower oleosin (Cummins, et al. (1992) Plant Mol. Biol. 19:873-876), and the like.

Examples of endosperm-specific promoters include, but are not limited to, rice prolamin NRP33 (Wu, et al. (1998) Plant Cell Physiol. 39(8):885-889); rice ADP-glucose pyrophosphorylase (Russell, et al. (1997) Trans. Res. 6:157-68); maize ESR gene family (Opsahl-Ferstad, et al. (1997) Plant J. 12:235-46); sorghum kafirin (DeRose, et al.

(1996) Plant Mol. Biol. 32:1029-35), and the like.

Examples of embryo-specific promoters include, but are not limited to, OSH1 (Sato, et al. (1996) Proc. Natl. Acad. Sci. USA 93:8117-8122); KNOX (Postma-Haarsma, et al. (1999) Plant Mol. Biol. 39:257-71); and PRO0151, PRO0175, PRO005 and PRO0095 (WO 2004/070039).

Examples of aleurone-specific promoters include, but are not limited to α-amylase (Lanahan, et al. (1992) Plant Cell 4:203-211); cathepsin β-like gene (Cejudo, et al. (1992) Plant Mol. Biol. 20:849-856); Barley Ltp2 (Kalla, et al. (1994) Plant J. 6:849-60); Chi26 (Leah, et al. (1994) Plant J. 4:579-89); Maize B-Peru (Selinger, et al. (1998) Genetics 149:1125-38) and the like.

A green tissue-specific promoter as defined herein is a promoter that is transcriptionally active predominantly in green tissue, substantially to the exclusion of any other parts of a plant, while still allowing for any leaky expression in these other plant parts. Examples of green tissue-specific promoters include, but are not limited to, leaf-specific Maize orthophosphate dikinase (Fukayama, et al. (2001) Plant Physiol. 127:1136-46); leaf-specific Maize phosphoenolpyruvate carboxylase (Kausch, et al. (2001) Plant Mol. Biol. 45:1-15); leaf-specific rice small subunit rubisco (Nomura, et al. (2000) Plant Mol. Biol. 44:99-106); rice beta expansin EXBP9 (WO 2004/070039); Pigeonpea small subunit rubisco (Panguluri, et al. (2005) Indian J. Exp. Biol. 43:369-72) and the like.

Those skilled in the art will be aware that an “inducible promoter” is a promoter the transcriptional activity of which is increased or induced in response to a developmental, chemical or physical stimulus. Preferred chemically-inducible promoters include the 3-β-indoylacrylic acid-inducible Tip promoter; IPTG-inducible lac promoter; phosphate-inducible promoter; L-arabinose-inducible araB promoter; heavy metal-inducible metallothionine gene promoter; dexamethasone-inducible promoter; glucocorticoid-inducible promoter; ethanol-inducible promoter (Zeneca); or any one or more of the chemically-inducible promoters described by Gatz, et al. (1998) Trends Plant Sci. 3:352-58, amongst others.

Preferred wound-inducible or pathogen-inducible promoters include the phenylalanine ammonia lyase (PAL) gene promoter (Ebel, et al. (1984) Arch. Biochem. Biophys. 232:240-248), chalcone synthase gene promoter (Ebel, et al. (1984) supra) or the potato wound-inducible promoter (Cleveland, et al. (1987) Plant Mol. Biol. 8:199-207), amongst others.

A developmentally-regulated promoter is active during certain developmental stages or in parts of the plant that undergo developmental changes.

In addition to a promoter, constructs for overexpressing MdMYB3 can also include other regulatory elements. Such elements include, but are not limited to, enhancers, silencers, intron sequences, 3′UTR and/or 5′UTR regions and/or RNA stabilizing elements. The term “terminator” encompasses a regulatory element, which is a DNA sequence at the end of a transcriptional unit that signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene.

If polypeptide expression is desired, it is generally desirable to include a polyadenylation region at the 3′ end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman & Berg (1988) Mol. Cell. Biol. 8: 4395-4405; Callis, et al. (1987) Genes Dev. 1:1183-1200). Such intron enhancement of gene expression is typically greatest when placed near the 5′ end of the transcription unit. Use of the Adhl-5 intron or the Bronze-1 intron is known in the art. For general information see The Maize Handbook, Chapter 116, Freeling & Walbot, Eds., Springer, NY (1994).

In other embodiments, the levels of MdMYB3 are decreased thereby decreasing the levels of anthocyanin and decreasing the length of peduncles and styles in plants. Reference herein to “decreased expression” refers to a decrease in endogenous gene expression and/or polypeptide levels and/or polypeptide activity relative to control plants. Such a decrease is at least a 10%, 20%, 30%, 40% or 50%, 60%, 70%, 80%, 85%, 90%, or 95%, 96%, 97%, 98%, 99% or more decrease compared to that of control plants.

A decrease in the expression of endogenous MdMYB3 can be carried out by gene knock out or gene silencing using routine tools and techniques. Typically, gene silencing methods include the use of nucleic acid molecules that are complementary to the nucleic acid molecule encoding MdMYB3 thereby inhibiting or blocking the expression thereof. One such method for the reduction of endogenous gene expression is RNA-mediated silencing of gene expression (downregulation). Silencing in this case is triggered in a plant by a double-stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double-stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence includes a nucleotide sequence that is complementary to a “sense” nucleic acid sequence encoding a protein, i.e., complementary to the coding strand of a double-stranded cDNA molecule or complementary to an mRNA transcript sequence. The antisense nucleic acid sequence is preferably complementary to the endogenous gene to be silenced. The complementarity may be located in the coding region and/or in the non-coding region of a gene. The term coding region refers to a region of the nucleotide sequence comprising codons that are translated into amino acid residues. The term non-coding region refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid capable of encoding an orthologue, paralogue or homologue of the protein of interest), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct include a promoter (as described here), an operably linked antisense oligonucleotide, and a terminator (as described here).

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. The antisense nucleic acid sequences can also be delivered to cells using the vectors described herein.

A decrease in the expression of MdMYB3 may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes described in Haselhoff & Gerlach (1988) Nature 334:585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see, for example, U.S. Pat. No. 4,987,071 and U.S. Pat. No. 5,116,742). Alternatively, mRNA transcripts corresponding to a nucleic acid sequence can be used to select a catalytic RNA having a specific ribonuclease activity from a pool of RNA molecules (Bartel & Szostak (1993) Science 261:1411-1418). The use of ribozymes for gene silencing in plants is known in the art (e.g., WO 94/00012; WO 95/03404; WO 00/00619; WO 97/13865 and WO 97/38116).

Artificial and/or natural microRNAs (miRNAs) may also be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. mRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically nucleotides in length, can be genetically engineered specifically to negatively regulate gene expression of single or multiple genes of interest. Determinants of plant microRNA target selection are well-known in the art. Empirical parameters for target recognition have been defined and can be used to aid in the design of specific amiRNAs, (Schwab, et al. (2005) Dev. Cell 8:517-527). Convenient tools for design and generation of amiRNAs and their precursors are also available to the public (Schwab, et al. (2006) Plant Cell 18:1121-1133).

Gene silencing may also be achieved by insertion mutagenesis (for example, T-DNA insertion or transposon insertion) or by strategies as described by, among others, Angell & Baulcombe ((1999) Plant J. 20(3):357-62), WO 98/36083, or WO 99/15682.

A further approach to gene silencing is by targeting nucleic acid sequences complementary to the regulatory region of the gene (e.g., the promoter and/or enhancers) to form triple helical structures that prevent transcription of the gene in target cells. See, Helene (1991) Anticancer Drug Res. 6:569-84; Helene, et al. (1992) Ann. N.Y. Acad. Sci. 660:27-36 1992; and Maher (1992) Bioassays 14:807-15.

Other methods, such as the use of antibodies directed to an endogenous polypeptide for inhibiting its function in planta, or interference in the signaling pathway in which a polypeptide is involved, will be well known to the skilled artisan. In particular, it can be envisaged that man-made molecules may be useful for inhibiting the biological function of a target polypeptide, or for interfering with the signaling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the decrease in expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

When introducing nucleic acids into plants for increasing or decreasing expression of MdMYB3, it is typically desirable that the nucleic acids are provided in an expression construct to facilitate manipulation, replication (e.g., in a bacterial cell) and introduction into plants. Accordingly, this invention also includes a vector, preferably an expression vector, harboring nucleic acids encoding MdMYB3 or a gene silencing construct thereto. Vectors of this invention can include one or more selectable markers (gene) and/or reporter genes that confer a phenotype on a cell in which it is expressed to facilitate the identification and/or selection of cells that are transfected or transformed with a nucleic acid construct of the invention. Marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to BASTA; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilization of xylose, or anti-nutritive markers such as the resistance to 2-deoxyglucose). Expression of visual marker genes results in the formation of color (for example β-glucuronidase, GUS or β-galactosidase with its colored substrates, for example X-Gal), luminescence (such as the luciferin/luciferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that includes the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, includes (up to 40% or more of the transformants), both vectors. In case of transformation with Agrobacteria, the transformants usually receive only a part of the vector, i.e., the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Crel is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble, et al. (2000) J. Biol. Chem. 275:22255-22267; Velmurugan, et al. (2000) J. Cell Biol. 149:553-566). A site-specific integration into the plant genome of the nucleic acid sequences according to the invention is possible. Naturally, these methods can also be applied to microorganisms such as yeast, fungi or bacteria.

Introduction or transformation of a vector or nucleic acid of the invention encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, et al. (1982) Nature 296:72-74); electroporation of protoplasts (Shillito, et al. (1985) Bio/Technol. 3:1099-1102); microinjection into plant material (Crossway, et al. (1986) Mol. Gen. Genet. 202:179-185); DNA or RNA-coated particle bombardment (Klein, et al. (1987) Nature 327:70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. The plant is subsequently grown until the seeds of the treated plant are obtained (Clough & Bent (1998) Plant J. 16:735-743). Methods for Agrobacterium-mediated transformation of rice include well-known methods for rice transformation, such as those described in EP 1198985 A1, Aldemita & Hodges (1996) Planta 199:612-617; Chan, et al. (1993) Plant Mol. Biol. 22(3):491-506; Hiei, et al. (1994) Plant J. 6(2):271-282. In the case of corn transformation, the preferred method is as described by either Ishida, et al. (1996) Nat. Biotechnol. 14(6):745-50 or Frame, et al. (2002) Plant Physiol. 129(1):13-22. The nucleic acids or the construct to be expressed is preferably cloned into a vector, which is suitable for transforming Agrobacterium tumefaciens, for example pEin19 (Bevan, et al. (1984) Nucl. Acids Res. 12:8711) or, as described herein, pBI121. Agrobacteria transformed by such a vector can then be used in known manner for the transformation of plants, such as plants used as a model, like Arabidopsis or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen & Willmitzer (1988) Nucl. Acid Res. 16:9877.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic (Feldman & Marks (1987) Mol. Gen. Genet. 208:274-289). Alternative methods are based on the repeated removal of the inflorescences and incubation of the excision site in the center of the rosette with transformed agrobacteria, whereby transformed seeds can likewise be obtained at a later point in time (Chang (1994) Plant J. 5:551-558; Katavic (1994) Mol. Gen. Genet. 245:363-370). However, an especially effective method is the vacuum infiltration method with its modifications such as the “floral dip” method. In the case of vacuum infiltration of Arabidopsis, intact plants under reduced pressure are treated with an agrobacterial suspension (Bechthold (1993) C R Acad. Sci. Paris Life Sci. 316:1194-1199), while in the case of the “floral dip” method, the developing floral tissue is incubated briefly with a surfactant-treated agrobacterial suspension (Clough & Bent (1998) Plant J. 16:735-743). A certain proportion of transgenic seeds are harvested in both cases, and these seeds can be distinguished from non-transgenic seeds by growing under the above-described selective conditions. In addition, the stable transformation of plastids is of advantages because plastids are inherited maternally is most crops reducing or eliminating the risk of transgene flow through pollen. The transformation of the chloroplast genome is generally achieved by a process which has been schematically displayed by Klaus, et al. (2004) Nature Biotechnology 22:225-229. Briefly, the sequences to be transformed are cloned together with a selectable marker gene between flanking sequences homologous to the chloroplast genome. These homologous flanking sequences direct site-specific integration into the plastome. Plastidal transformation has been described for many different plant species and an overview is given in Bock (2001) J. Mol. Biol. 312(3):425- or Maliga (2003) Trends Biotechnol. 21:20-28. Further biotechnological progress has been reported in form of marker-free plastid transformants, which can be produced by a transient co-integrated maker gene (Klaus, et al. (2004) Nature Biotechnology 22(2):225-229).

Introduction or transformation of plants with nucleic acids disclosed herein results in transgenic plants harboring expression constructs for decreasing or increasing the expression of MdMYB3 in said transgenic plant. Accordingly, this invention also provides such transgenic plants, including whole plants, ancestors and progeny of the plants and plant parts, including seeds, shoots, stems, leaves, roots (including tubers), flowers, and tissues and organs, wherein each of the aforementioned include the nucleic acid of interest. The term “plant” also encompasses plant cells, suspension cultures, callus tissue, embryos, meristematic regions, gametophytes, sporophytes, pollen and microspores, again wherein each of the aforementioned includes the nucleic acid of interest.

Plants that are particularly useful in this invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g., Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g., Brassica napes, Brassica rapa ssp. (canola, oilseed rape, turnip rape)), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., Elaeis guineensis, Elaeis oleifera), Eleusine coracana, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g., Glycine max, Soja hispida or Soja max), Gossypium hirsutum, Helianthus spp. (e.g., Helianthus annuus), Hemerocallis fulva, Hibiscus spp., Hordeum spp. (e.g., Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g., Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

Transgenic plants of this invention exhibit an increase or decrease in the expression of MdMYB3 and resulting increase or decrease in accumulation of anthocyanins and length of peduncles and styles. Accordingly, this invention also provides a method for modulating the color (e.g., anthocyanins and flavonols) and/or length of peduncles and/or styles by modulating the expression or activity of MdMYB3. In one embodiment, the accumulation of anthocyanins and flavonols is increased or enhanced as compared to a control or wild-type plant by overexpressing MdMYB3. This embodiment finds application in producing plants or plant parts with enhanced or increased red, red, purple, or blue coloration. Such plants can have enhanced appeal, improved neuroprotective and anti-inflammatory activities (Korte, et al. (2009) J. Med. Food 12:1407-10), monoamine oxidase inhibitory activity (Dreiseitel, et al. (2009) Pharmacol. Res. 59:306-11) and cancer preventive activity.

In another embodiment, the accumulation of anthocyanins and flavonols is decreased or reduced as compared to a control or wild-type plant by inhibiting the expression or activity of MdMYB3, or paralogs or orthologs thereof. This embodiment finds application in producing plants or plant parts with reduced or decreased red, red, purple, or blue coloration, e.g., in ornamental applications.

In another embodiment, the length of peduncles and/or styles is increased or enhanced as compared to a control or wild-type plant by overexpressing MdMYB3. Plants with increased peduncle length can be used in ornamental applications or to facilitate fruit harvest (e.g., pumpkin, squash or strawberry production). Likewise, plants with increased style lengths can be used in ornamental applications or to modulate pollination. In particular, cross hybridization can be challenging in plant species that have short styles (e.g., rice), thereby making it difficult to develop hybrid varieties. For example, cross pollination in rice is less than 1%. As such, efforts to develop hybrid rice varieties with improved characteristics such as yield, grain size, disease resistance, among others, are quite difficult. Overexpression of MdMYB3 gene in rice can elongate the style-stigma of the flower, thereby contributing to higher frequencies of successful cross pollination in rice.

In still another embodiment, the length of peduncles and/or styles is decreased or reduced as compared to a control or wild-type plant by inhibiting the expression or activity of MdMYB3, or paralogs or orthologs thereof. Plants with decreased peduncle length and/or style length can be used, e.g., in ornamental applications or to modulate pollination.

For the purposes of the present invention, the terms “increase,” “improve” and “enhance” are interchangeable and refer to at least a 3%, 4%, 5%, 6%, 7%, 8%, 9% or 10%, preferably at least 15% or 20%, more preferably 25%, 30%, 35% or 40% more increase in accumulation of anthocyanins/flavonols and/or length of peduncles and/or length of styles in transgenic plants in comparison to control or wild-type plants. Likewise, the terms “decrease” and “enhance” are interchangeable and refer to at least a 3%, 4%, 5%, 6%, 7%, 8%, 9% or 10%, preferably at least 15% or 20%, more preferably 25%, 30%, 35% or 40% more decrease in accumulation of anthocyanins/flavonols and/or length of peduncles and/or length of styles in transgenic plants in comparison to control or wild-type plants.

The invention is described in greater detail by the following non-limiting examples.

### Example 1

### Materials and Methods

Plant Material.

Leaves, flowers, and fruits at different stages of development were collected from trees of apple cultivars (cvs.) Red Delicious and Golden Delicious. In addition, fruits of cvs. Baihaitang, Gala, Huangtaiping, Jinhong, Dolgo, Jiguan, Golden Delicious, Sompain, Baishaguo, and Mutsu were also collected at maturity.

Arabidopsis AtMYB3 (AT1G22640) was compared against an apple EST database, and a homologous EST contig (accession no. Apple_{—}0223.1923.C1.Contig3505) was identified. The EST contig sequence was then compared against the apple EST database of NCBI, and an EST sequence (GenBank accession no. 00868594) containing both R2 and R3 domains was recovered. Based on the EST sequence, a pair of primers (5′-GGG AGA GCA CCT TGT TGT GAG-3′, SEQ ID NO:10; 5′-GAT CTC GTT GTC GGT TCT TCC-3′, SEQ ID NO:11) was then designed and used to screen BAC library of cv. GoldRush using a PCR-based screening method (Xu, et al. (2002) Acta Hort. 595:103-112). The reaction was carried out as follows: 94° C. for 3 minutes, followed by 33 cycles of 94° C. for 35 seconds, 55° C. for 30 seconds, 72° C. for 60 seconds, and followed by a final 8 minute extension at 72° C. A positive BAC clones was randomly selected and subjected to sequencing to recover genomic sequence encoding MdMYB3 in apple.

Recovery of cDNA Sequence Encoding MdMYB3 in Apple.

Genomic sequences encoding MdMYB3 were analyzed using FGENESH-M program, and an open reading frame (ORF) was predicted. A pair of primers (5′-GGA GAG CAC CTT GTT GTG AG-3′, SEQ ID NO:12; 5′-ACT GAC AAT TGC TGC ATG CC-3′, SEQ ID NO:13) was designed based on the predicted ORF, and used to amplify cDNA from leaves of cv. GoldRush. The PCR product was sequenced, and a cDNA fragment 872 by in size was recovered. The cDNA fragment sequence was compared against NCBI EST database, and a cDNA containing the full coding region was identified. Subsequently, a pair of primers (5′-CTG ATC CAG AAG AAG AAA CAG ATG-3′, SEQ ID NO:14; 5′-TGG ATT CAA AGC AGG TCT GTG-3′, SEQ ID NO:15) was designed to amplify the full coding region of MdMYB3 from cv. GoldRush to further verify the predicted ORF.

Expression Vector Construction and Tobacco Transformation.

A pair of primers (5′-TGA CTC TAG ACT GAT CCA GAA GAA GAA AC-3′, SEQ ID NO:16; 5′-ATA CGA GCT CTG GAT TCA AAG CAG-3′, SEQ ID NO:17) was designed to amplify the coding region of MdMYB3 using the proofreading DNA polymerase PLATINUM Pfx (Invitrogen) following the manufacturer's instructions. Forward and reverse primers contained XbaI and Sad restriction sites at the 5′ end, respectively. The blunt-end PCR product was ligated into the PCR-Blunt vector using ZERO BLUNT PCR cloning kit (Invitrogen) according to the manufacturer's protocol. The expression vector was confirmed by direct sequencing. The coding sequence of MdMYB3 was introduced into the pBI121 cloning vector, and the construct was used for Agrobacterium-mediated transformation of tobacco (Nicotiana tabacum cv. Petite Havana SR1) according to established methods (Han, et al. (2011) Plant Physiol. 153:806-820). T1 seed from three confirmed independent transgenic TO lines overexpressing MdMYB3 and carrying a single copy of the transgene, including OE-1, OE-5, and OE-8 were selfed to generate T2 plants.

Wild-type and T2 transgenic tobacco plants were grown in the greenhouse, and flowers at full-bloom (completely open flowers) were collected for analysis of gene expression as well as for analysis of contents of flavonoid compounds. Upon collection, all samples were frozen in liquid nitrogen and stored at −80° C. until needed.

Mapping of the MdMYB3 Gene onto the Apple Linkage Map.

A simple sequence repeat (SSR) marker within a 5′ untranslated region of MdMYB3 was used to screen an F₁ mapping population derived from a cross between ‘Co-op 16’ and ‘Co-op 17’. The primer sequences of the SSR marker were as follows: forward 5′-TCA CCT CTT CAA ACA ACA CAC C-3′ (SEQ ID NO:18) and reverse 5′-TGC TCT CCC CAT CTG TTT CT-3′ (SEQ ID NO:19). The PCR product was separated on a 2% (w/v) METAPHOR agarose gel. The linkage map was constructed using JoinMap version 4.0, according to established methods (Han, et al. (2011) supra).

Real-Time PCR Analysis.

Total RNA from leaf and flower tissues were extracted using an RNAQUEOUS Kit (Ambion) according to the manufacturer's instructions. RNA from fruit tissues was isolated according to an established protocol (Gasic, et al. (2004) Plant Mol. Biol. Rep. 22:437a-437 g). Total RNA (2 μg) from each tissue was treated with DNaseI (Invitrogen), and used for cDNA synthesis. The first-strand cDNA synthesis was performed with Oligo (dT) primer using the SUPERSCRIPT III RT kit (Invitrogen), according to the manufacturer's instructions. Specific primers for MdMYB3 and each flavonoid-related gene were designed using Biology Workbench version 3.2. Specific primer sequences and accession numbers of genes used to design primers are listed in Table 1.

TABLE 1

| | | | | |
|---|---|---|---|---|
| Gene | Gene | | | |
| Name | Identifier | Primer | Sequence | SEQ ID NO: |

Malus

| | | | | |
|---|---|---|---|---|
| MdMYB4 | | Forward | GGCAAGAGTTGCAGGTTGAG | 20 |
| | | Reverse | GTCGCTTGATGTGTGTGTTCC | 21 |
| MdCHS | X68977 | Forward | TCAAGCCTATTGGGATTTCG | 22 |
| | | Reverse | CAGCTGACTTCCTCCTCACC | 23 |
| MdCHI | X68978 | Forward | GATATCGAAGCCGGAAATGA | 24 |
| | | Reverse | TGTTGACTCACGCCAACAAT | 25 |
| MdF3H | AF117270 | Forward | ACACCAAATATGGCTCCTGC | 26 |
| | | Reverse | TTTCGTTGCTGAAGTCGTTG | 27 |
| MdFLS | AF119095 | Forward | AATGGGAGTGGAGTCTGTGG | 28 |
| | | Reverse | AGTTGGAGCTGGCCTCAGTA | 29 |
| MdDFR | AF117268 | Forward | AAGGCCGTTACATTTGTTCG | 30 |
| | | Reverse | GCCCTTGAACTTTGTGGGTA | 31 |
| MdUFGT | AF117267 | Forward | AGCTCCACTCGGAACTTCAA | 32 |
| | | Reverse | AACCCGCCCTAAATATGTCC | 33 |
| MdANS | AF117269 | Forward | CAATTTGGCCTCAAACACCT | 34 |
| | | Reverse | TCAACACCAAGTGCAAGCTC | 35 |
| MdANR | DQ099803 | Forward | GTTGCAACCCCTGTCAACTT | 36 |
| | | Reverse | CACGACCAAACCTGTTCCTT | 37 |
| MdLAR1 | DQ139836 | Forward | ACAACACCCACCCTTCTGAG | 38 |
| | | Reverse | TGCAGCAAGGGCTAGTAGGT | 39 |
| MdActin | DQ822466 | Forward | CTACAAAGTCATCGTCCAGACAT | 40 |
| | | Reverse | TGGGATGACATGGAGAAGATT | 41 |

Nicotiana

| | | | | |
|---|---|---|---|---|
| NbActin | AY179605 | Forward | AATGATCGGAATGGAAGCTG | 42 |
| | | Reverse | TGGTACCACCACTGAGGACA | 43 |
| NtCHI | AB213651 | Forward | GAAATCCTCCGATCCAGTGA | 44 |
| | | Reverse | CAACGTTGACAACATCAGGC | 45 |
| NbCHS | EF421432 | Forward | AGAAAAGCCTTGTGGAAGCA | 46 |
| | | Reverse | ACTTGGTCCAAAATTGCAGG | 47 |
| NtF3H | AB289450 | Forward | ACAGGGTGAAGTGGTCCAAG | 48 |
| | | Reverse | CCTTGGTTAAGGCCTCCTTC | 49 |
| NtF3′H | AB289449 | Forward | TCCAAGAATACTGGCCCAAG | 50 |
| | | Reverse | CTCACAACTCTCGGATGCAA | 51 |
| NtFLS | AB289451 | Forward | GAACTTGAAGGGAAAAGGGG | 52 |
| | | Reverse | TCCCTGTAGGAGGGAGGATT | 53 |
| NbDFR1 | EF421431 | Forward | TCCCATCATGCGATCATCTA | 54 |
| | | Reverse | ATGGCTTCTTTGTCACGTCC | 55 |
| NtLAR | AM827419 | Forward | TCAAGGTCCTTTACGCCATC | 56 |
| | | Reverse | ACGAACCTGCTTCTCTTTGG | 57 |
| NtANS | AB289447 | Forward | TGGCGTTGAAGCTCATACTG | 58 |
| | | Reverse | TTTCAAGGGTGTCCCCAATA | 59 |
| NtUFGT | FG627024 | Forward | GAGTGCATTGGATGCCTTTT | 60 |
| | | Reverse | CCAGCTCCATTAGGTCCTTG | 61 |
| NtANR1 | AM791704 | Forward | CATTTGACTTTCCCAAACGC | 62 |
| | | Reverse | ATTGGGCTTTTGAGTTGTGC | 63 |
| NtANR2 | DW003895 | Forward | TGTTCCCACTTGGGATGATA | 64 |
| | | Reverse | TGCACCTATACTCTGTTAGTGGC | 65 |
| NtC4H | AB236952 | Forward | CCAGGAGTGCAAGTGACTGA | 66 |
| | | Reverse | ACCACCAAGCGTTAACCAAG | 67 |
| NtPAL | AB289452 | Forward | CCTCAGAACATCACCCCAGT | 68 |
| | | Reverse | ACCGTGTAACGCCTTGTTTC | 69 |
| NtCOMT | Z56282 | Forward | TTTTCGTGGATGCTGACAAG | 70 |
| | | Reverse | GGGTAATTCCATCACCAACG | 71 |
| NtCAD | AY911854 | Forward | CGAAGACATTGGCTGAGGAT | 72 |
| | | Reverse | TTGGGTATGTTTCAGCACCA | 73 |

### Artificial Sequence

The SYBR Green real-time PCR assay was carried out in a total volume of 25 μl, composed of 12.5 μL of 2×SYBR Green I Master Mix (Applied Biosystems), 0.2 μM (each) specific primers, and 100 ng of template cDNA. The amplification program included 1 cycle of 95° C. for 10 minutes, followed by 40 cycles of 95° C. for 15 seconds, and 60° C. for 1 minute. The fluorescent product was detected at the last step of each cycle. Following amplification, melting temperatures of PCR products were analyzed to determine specificity of the PCR product. Melting curves were obtained by slow-heating at 0.5° C./seconds, from 60° C. to 90° C., while continuously monitoring the fluorescence signal. A negative control without a cDNA template was run with each analysis to evaluate the overall specificity. Amplifications were carried out in a 96-well plate in a 7300 Real Time PCR System (Applied Biosystems). All experimental samples were run in triplicate. An apple Actin gene was used as a constitutive control. Differences between the cycle threshold (Ct) of the target gene and the Actin gene were used to obtain relative transcript levels of the target gene, and calculated as 2 exp−(Ct_{target}−Ct_{actin}) .

Flavonoid Analysis. Anthocyanins and flavonols were extracted from 50 mg of finely ground tissue in 1 ml 1% HCl/methanol (v/v), at room temperature in the dark, with continuous shaking for 1 hour, and centrifuged at 13,000 rpm for 15 minutes. An aliquot of 100 μL of the supernatant was transferred to a fresh tube, and acid-hydrolyzed by adding 30 μL of 3N HCl, and incubated at 70° C. for 1 hour in a thermal cycler (Thermo Hybaid MBS 0.25s, Thermo Scientific). Proanthocyanins (PAs) were extracted using 1 ml 70% (v/v) acetone containing 0.1% (w/v) ascorbate, and incubated at room temperature for 24 hours in darkness (Takos, et al. (2006) supra). The extract was centrifuged at 13,000 rpm for 15 minutes at room temperature, and the clear supernatant was transferred to a new tube. An aliquot of 200 μL extract was dried at 35° C., and resuspended in 100 μL of 1% (v/v) HCl-methanol and 100 μL of 200 mM sodium acetate (pH 7.5).

Flavonoid contents were determined using LC/MS/MS along with use of commercial standards for kaempferol, quercetin, cyanidin, catechin, and epicatechin (Sigma). The LC/MS/MS analysis was performed on a 5500 QTRAP mass spectrometer (AB Sciex) equipped with a 1200 Agilent HPLC Analyst (version 1.5.1, Applied Biosytems) for data acquisition and processing. A Phenomenex column (3μ C6-Phenyl) 11A, 4.6×50 mm) was used for separation. The HPLC flow rate was set at 0.3 mL/min, and HPLC mobile phases included A (0.1% formic acid in H₂O) and B (0.1% formic acid in acetonitrile). The autosampler was maintained at 5° C. The gradient for catechin and epicatechin was as follows: 0 minute, 90% A; 10 minutes, 50% A; 13-18 minutes, 0% A; and 18.1-25 minutes, 90% A. The injection volume was 20 μl. The gradient for cyanidin, kaempferol, and quercetin was as follows: 0 minute, 70% A; 7-12.5 minutes, 0% A; and 13-20 minutes, 70% A. The injection volume was 10 μL. The mass spectrometer was operated with positive electrospray ionization. Multiple reaction monitoring (MRM) was used to quantify catechin and epicatechin (m/z 291.0->139.2), cyanidin (m/z 287.2->213.2), kaempferol (m/z 287.1->153.2), and quercetin (m/z 303.1->153.1). The electrospray voltage was set to 5500 V; the heater was set at 600° C.; the curtain gas was at 35 psi; and GS1 and GS2 were both at 60 psi. Analysis of each sample was repeated three times using three biological replicates.

### Example 2

### Sequence Characterization of MdMYB3 in Apple

A genomic DNA sequence encoding R2R3MYB was isolated from cv. GoldRush. When this sequence was compared against the Arabidopsis genome sequence database, a best hit to the AtMYB3 gene was found, and thus the gene was designated as MdMYB3. The MdMYB3 gene was composed of three exons and two introns along with two tandem repeats, (TC)₁₆(TA)₁₂ (SEQ ID NO:74), designated as SSRI in the 5′ untranslated region (UTR), as well as a dinucleotide (GT)₅ (SEQ ID NO:75), designated as SSR2 in the last exon. The full-length cDNA of MdMYB3 was 1193 by in size and encoded a putative protein of 310 amino acids along with an ATG start codon, at position 162 of the nucleotide sequence, and a TGA stop codon, at position 1094.

Phylogenetic analysis based on amino acid sequences of R2R3MYB encoding genes from different plants indicated that MdMYB3 was very closely related to Arabidopsis AtMYB3, AtMYB4, and AtMYB7, belonging to the subgroup 4 R2R3 family of plant MYB transcription factors (Kranz, et al. (1998) Plant J. 16:263-276). Amino acid sequence alignment between MdMYB3 and several previously reported MYB transcription factors, including Arabidopsis AtMYB3, AtMYB4, AtMYB7, and AtMYB32, Fragaria ananasa FaMYB1, and Zea mays ZmMYB31 revealed that MdMYB3 was composed of both R2 and R3DNA-binding domains (FIG. 1). An R/B-like bHLH binding motif, [D/E]Lx₂[R/K]x₃Lx₆Lx₃R (SEQ ID NO:76) (Zimmermann, et al. (2004) Plant J. 40:22-34), was identified in the R3-DNA binding domain of MdMYB3 (FIG. 1). Moreover, MdMYB3 contained two conserved motifs LIsrGIDPx^{T}/ₛHRx^{I}/_{L} (C1-motif; SEQ ID NO:8) and pdLNL^{D}/_{E}Lxi^{G}/_{S} (C2-motif; SEQ ID NO:9) at the C-terminus, previously found in R2R3MYB encoding genes of subgroup 4. However, the C-terminal downstream of the two conserved motifs showed high divergence. MdMYB3 has a 50% amino acid sequence identity with AtMYB3. The nucleotide and amino acid sequence of MdMYB3 are set forth herein as SEQ ID NO:1 and SEQ ID NO:2, respectively.

### Example 3

### Mapping of the MdMYB3 Gene onto the Apple Genetic Map

Two pairs of primers flanking the SSRI and SSR2 repeats within the MdMYB3 gene were designed and used to screen the two parents of the F₁ population of the ‘Co-op 16’×‘Co-op 17’ cross. The two parents were found to be heterozygous and homozygous at SSR1 and SSR2 loci, respectively. The primers flanking the SSRI locus were then selected to screen F₁ progenies of ‘Co-op 16’×‘Co-op 17’ cross. As a result, three genotypes, designated ‘hh’ (upper band), ‘hk’ (upper and lower bands), and ‘kk’ (lower band), respectively, were identified for the SSRI locus among this progeny. Based on an apple genetic linkage map (Han, et al. (2011) supra), the apple MdMYB3 gene was anchored onto linkage group 15.

### Example 4

### Expression Profile of MdMYB3 in Apple

Expression profiles of MdMYB3 in apple cvs. Red Delicious (red-skinned fruit) and Golden Delicious (yellow-skinned fruit) were investigated. Quantitative real-time (qRT)-PCR analysis revealed that MdMYB3 transcripts accumulated in all analyzed tissues, including leaves, flowers, and fruits (FIG. 3). Overall, transcript levels of MdMYB3 in all analyzed tissues were higher in cv. Red Delicious than those in cv. Golden Delicious. Accumulation of MdMYB3 transcripts in flowers of ‘Red Delicious’ increased throughout flower development and reached a peak at full-bloom (completely open flowers), while transcripts of MdMYB3 in flowers of ‘Golden Delicious’ showed a peak at the balloon stage (closed, yet ballooned flower buds), and then slightly decreased until full-bloom (fully-open flowers). Transcripts of MdMYB3 in fruits of both cvs. Red Delicious and Golden Delicious increased during early stages of development, but then decreased slightly at 44 days after pollination (DAP). Subsequently, transcript accumulation of MdMYB3 in fruits of cv. Golden Delicious gradually increased until maturity; whereas, those of cv. Red Delicious peaked at fruit stage 1V, and remained relatively high at fruit maturity.

Subsequently, a total of 10 apple cultivars were selected and used to investigate the association of MdMYB3 gene expression with anthocyanin accumulation in excocarp of fruits at maturity. Overall, MdMYB3 transcripts were highly expressed in excocarp of red-skinned fruits, but were either low or undetectable in yellowish-green skinned fruits. These expression profiles were accompanied with similar anthocyanin content profiles in cortex tissues of these apple cultivars. This finding further confirmed that MdMYB3 was involved in anthocyanin accumulation in apple.

### Example 5

### Functional Analysis of MdMYB3 in Tobacco

The coding sequence of MdMYB3, driven by the constitutive promoter of cauliflower mosaic virus (CaMV) 35S, was introduced into tobacco, and three T₂ transgenic lines, designated as 0E-1, OE-5, and OE-8, were generated. Flowers of transgenic lines showed darker color pigmentation than those of wild-type plants. For example, corolla of flowers of plants of line OE-5 began to show pink coloration during earlier stages of flower development than those of wild-type plants. Subsequently, at early bloom, corollas of flowers of OE-5 were almost dark pink while those of wild-type were light pink. Corolla of flowers of all three transgenic lines continued to show increased pigmentation until full-bloom (completely open flowers), and showed markedly darker pink coloration, almost fuchsia, than those of wild-type plants.

LC/MS/MS analysis revealed that transgenic flowers of tobacco contained higher levels of flavonoids than wild-type flowers (Table 2).

TABLE 2

| | | | | Proanthocyanidin | |
|---|---|---|---|---|---|
| | | | Anthocya- | (ng/g) | |
| | Flavonol (ng/g) | | nidin (ng/g) | | Epica- |
| Flower | Kaempferol | Quercetin | Cyanidin | Catechin | techin |
| WT | 60.57 | 71.80 | 1722 | 4.80 | 6.47 |
| MdMYB3-1 | 53.93 | 88.83 | 3060 | 5.60 | 8.60 |
| MdMYB3-5 | 73.50 | 136.67 | 7707 | 6.20 | 29.13 |
| MdMYB3-8 | 79.27 | 114.67 | 4513 | 6.93 | 18.93 |

*Kaempferol, quercetin, cyanidin, catechin, and epicatechin were used as standards. All data correspond to mean values of the three biological replicates.

For example, levels of cyanidin in transgenic flowers were 2- to 4-fold higher than those of wild-type flowers. Moreover, levels of two proanthocyanidin components, catechin and epicatechin, in transgenic flowers were 1.1- to 1.4-fold and 1.3- to 4.5-fold, respectively, higher than those of wild-type flowers. These findings indicated that MdMYB3 was involved in the regulation of flavonoid biosynthesis in tobacco flowers.

In addition to flower color pigmentation, differences in other morphological traits, including lengths of flowers and lengths of styles of pistils, were also observed between wild-type and transgenic lines. For example, at full bloom, flowers of tobacco plants of transgenic line OE-5 were longer, on average 8-10 mm longer, than those of wild-type plants. Moreover, lengths of styles of pistils of transgenic flowers were also longer, on average 10-14 mm longer, than those of wild-type flowers, thus positioning stigmas above anthers.

### Example 6

### Expression Profile of Structural Genes of Phenylpropanoid and Flavonoid Pathways in Tobacco Transgenic Flowers Overexpressing MdMYB3

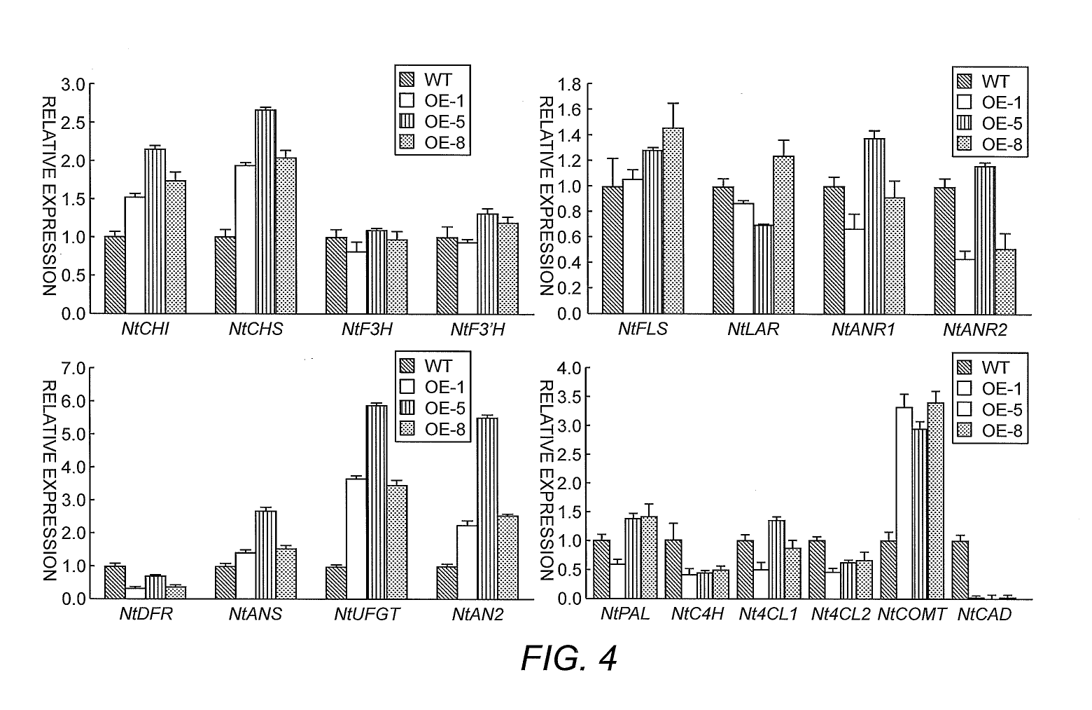

Transcripts of 15 structural genes involved in biosynthesis pathways of both phenylpropanoid and flavonoid were evaluated in flowers of both wild-type and transgenic tobacco plants (FIG. 4). Of these 15 genes, flavonoid-specific genes, including NtCHI, NtCHS, NtANS, NtUFGT, NtAn2, and NtCOMT showed similar patterns in transcript accumulation for all three transgenic lines as they were significantly up-regulated compared to those of wild-type flowers (FIG. 4). Moreover, transcripts of NtDFR and three phenylpropanoid pathway genes including NtC4H, Nt4CL2, and NtCAD exhibited similar patterns of gene expression as they were all down-regulated in flowers of all three transgenic lines compared to those of wild-type plants. All remaining genes showed different patterns of gene expression in flowers transgenic lines when compared to those wild-type plants.

What is claimed is:

1. A purified nucleic acid molecule comprising:
(a) a nucleotide sequence encoding a transcription factor having the amino acid sequence of SEQ ID NO:2;
(b) the nucleotide sequence of SEQ ID NO:1 or a sequence at least 75% identical thereto; or
(c) a nucleotide sequence complementary to a nucleotide sequence of (a) or (b).

2. A recombinant vector comprising the purified nucleic acid molecule of claim 1.

3. The recombinant vector of claim 2, wherein said vector is an expression vector.

4. A purified transcription factor polypeptide having the amino acid sequence of SEQ ID NO:2, or a polypeptide at least 75% identical thereto.

5. A transgenic plant or plant part comprising the purified nucleic acid molecule of claim 1.

6. A progeny of the transgenic plant of claim 5.

7. A seed of the transgenic plant of claim 5.

8. A method of modifying the color, length of peduncles, or length of styles of a plant comprising introducing into a plant the expression vector of claim 3 to produce a transgenic plant that exhibits modified color, length of peduncles, or length of styles when compared to a control plant.
